(19) United States
(12) Patent Application Publication
(10) Pub. No.: US 2018/0280565 A1
(43) Pub. Date: Oct. 4, 2018

(54) METHOD FOR ATTACHING BODY TISSUES AND KIT FOR PREPARING TISSUE ADHESIVE USED FOR THE SAME, AND DEVICE FOR TISSUE ADHESION

(71) Applicant: TERUMO KABUSHIKI KAISHA, Tokyo (JP)

(72) Inventor: Kunio FUKUI, Nakai-machi (JP)

(73) Assignee: TERUMO KABUSHIKI KAISHA, Tokyo (JP)

(21) Appl. No.: 15/936,794

(22) Filed: Mar. 27, 2018

(30) Foreign Application Priority Data
Mar. 29, 2017 (JP) 2017-066141

(51) Int. Cl. A61L 24/10; A61L 24/00; A61K 38/36; A61L 26/00; C09J 189/04; A61K 38/48; C08J 3/28

(52) U.S. Cl. CPC A61L 24/106; A61L 24/0042; A61K 38/363; C08J 3/28; C09J 189/04; A61K 38/4833; A61L 26/0042

(57) ABSTRACT

To provide a means which can suppress a decrease in the effect of preventing enzyme leakage and a decrease in adhesive strength (pressure resistance) when attaching body tissues by using a tissue adhesive containing a protein.

A method for attaching body tissues by applying a tissue adhesive containing a protein to the body tissues includes a heat treatment of the protein simultaneously with or after applying the tissue adhesive to the body tissues.

### CROSS-REFERENCE TO RELATED APPLICATION

The present application claims priority under 35 U.S.C. § 119 to Japanese Patent Application No. 2017-066141, filed Mar. 29, 2017. The contents of these applications are incorporated herein by reference in their entirety.

### BACKGROUND

### 1. Technical Field

The present invention relates to a method for attaching body tissues and a kit for preparing a tissue adhesive used for the same, and a device for tissue adhesion.

### 2. Description of Related Arts

Gastrointestinal fistulae are diseases in which an opening is developed in a gastrointestinal tract and the opening reaches the outside of the body, and are refractory diseases ranging a wide range of diseases such as inflammatory bowel diseases (e.g. Crohn disease, ulcerative colitis), postoperative suture insufficiency, etc. (e.g. enterocutaneous fistula, bile fistula, pancreatic fistula), post-radiotherapy complications (e.g. rectovaginal fistula, rectovesical fistula).

Among these, pancreatic fistula is a condition in which a fistulous opening is developed in pancreas and pancreatic fluid is continuously or intermittently leaked, and is one of the postoperative complications developed after pancreatic operations with high frequency. In addition, pancreatic fistula can be developed by pancreatitis (acute or chronic) or traumas.

As a method for preventing or treating a gastrointestinal fistula such as a pancreatic fistula caused by postoperative suture insufficiency, methods for example contact suture at the time of suture and indwelling of pancreatic duct stent, and moreover applying a tissue adhesive to block a fistulous tract with a gastrointestinal fistula to an affected part are carried out. As such tissue adhesive, the so-called “fibrin glue” and moreover those which contain a protein as a main component are widely used. Among these, the “fibrin glue” can be obtained by mixing two solutions, one of which mainly contains fibrinogen and the other of which mainly contains thrombin. Fibrinogen, a monomer, is polymerized by mixing due to the catalytic action of thrombin to form fibrin, a polymer, which acts as a glue to adhere a body tissue. The fibrin glue has good biological compatibility and has an advantage in being able to be indwelled in a living body, and thus is often used for operations. It should be noted that fibrin glue applied to an affected part is gradually decomposed and reduced in a living body, and finally absorbed into the body (see e.g. JP 02-071747 A and JP 60-204725 A).

### SUMMARY

Pancreatic fluid contains trypsin and chymotrypsin, which are digestive enzymes (endopeptidase) cleaving polypeptide forming a protein. Therefore, when these digestive enzymes leaked from a fistulous tract with pancreatic fistula are brought into contact with a protein (such as fibrin), the main component of a tissue adhesive, a digestive action on the protein is shown. As a result, there are problems in that the effect of preventing enzyme leakage by a tissue adhesive is not sufficiently obtained, and an adhesive strength (pressure resistance) of a fibrin glue decreases. As the handling of the problems, for example a method in which a trypsin inhibitor such as aprotinin is added to a tissue adhesive such as fibrin glue has been considered. The effect by this handling is however limited and another solution has been further demanded in the circumstances.

Therefore, an object of the present invention is to provide a means which can suppress a decrease in the effect of preventing enzyme leakage and a decrease in adhesive strength (pressure resistance) when attaching body tissues by using a tissue adhesive containing a protein.

The present inventors diligently investigated to solve the above problems. As a result, it was surprisingly found that a decrease in the effect of preventing enzyme leakage of the adhesive and a decrease in adhesive strength (pressure resistance) thereof could be suppressed by heating a protein, the main component of the adhesive, with a tissue adhesive applied to a body tissue, thereby completing the present invention.

That is, according to one aspect of the present invention, there is provided a method for attaching body tissues by applying a tissue adhesive containing a protein to the body tissues. The method is characterized by including a heat treatment of a protein contained in a tissue adhesive simultaneously with or after applying the tissue adhesive to body tissues.

### BRIEF DESCRIPTION OF THE DRAWINGS

FIG. 1 is a schematic to illustrate a device for tissue adhesion.

FIG. 2 contains photographs to show the experimental results in Example 1 described below.

FIG. 3 contains photographs to show the experimental results in Example 2 described below.

FIG. 4 contains photographs to show the experimental results in Example 3 described below.

### DETAILED DESCRIPTION

The embodiments of the present invention will now be described with reference to the attached drawings.

<>Method for Attaching Body Tissues<<

As described above, an aspect of the present invention relates to a method for attaching body tissues by applying a tissue adhesive containing a protein to the body tissues, the method including a heat treatment of the protein simultaneously with or after applying the tissue adhesive to the body tissues. This aspect will now be described in more detail.

[Tissue Adhesive]

A tissue adhesive is an agent used to attach body tissues. The tissue adhesive according to the present aspect contains a protein, and preferably contains a protein as a main component (50% by mass or more as the solid content concentration). The percentage of protein contained to the solid content (mainly fibrinogen and thrombin) concentration in a tissue adhesive is more preferably 30% by mass or more and further preferably 40% by mass or more.

As the tissue adhesive containing a protein, the so-called “fibrin glue”, which contains fibrin as the protein, is preferably used. The fibrin glue is a paste-like dispersion liquid obtained by dispersing fibrin in a medium such as water. It should be noted that the term “fibrin” as used herein is a concept which also covers a “fibrin clot”, in which fibrin is cross-linked by the action of e.g. blood coagulation factor VIII.

As commercially available fibrin glues, for example “BOLHEAL (registered trademark) for tissue adhesion” and “Beriplast (registered trademark) P Combi-Set for tissue adhesion” are known. In these commercially available fibrin glues, a composition containing fibrinogen and a composition containing thrombin are provided as lyophilizates in separate vials, and respective solutions obtained by further dissolving these components in dissolving liquids provided in separate vials before using are applied to an affected part. The method for attaching body tissues according to the present aspect also uses commercially available fibrin glue described above as a tissue adhesive in one of the preferred embodiments. That is, it is preferred that the method for attaching body tissues according to the present aspect further includes generating the fibrin by contact between

a first composition containing fibrinogen, and

a second composition containing thrombin.

It should be noted that “fibrinogen” as used herein is one which is normally considered as “fibrinogen” and is not particularly restricted as long as a tissue adhesive, an object of the present aspect, can be obtained. Fibrinogen provided by the above-described commercially available fibrin glues can be suitably used. In addition, as fibrinogen, a commercially available undiluted solution can be directly used, and an aqueous liquid produced by dissolution and/or dispersion using a proper aqueous medium can be also used. In addition, “thrombin” as used herein is one which is normally considered as “thrombin” and is not particularly restricted as long as a tissue adhesive, an object of the present aspect, can be obtained. Thrombin provided by the above-described commercially available fibrin glues can be suitably used. As thrombin, a commercially available undiluted solution can be directly used, and an aqueous liquid produced by dissolution and/or dispersion using a proper aqueous medium can be also used.

The “aqueous medium” as used herein is a medium which is normally used to produce a tissue adhesive and is formed from mainly water, and is not particularly restricted as long as a tissue adhesive, an object of the present aspect, can be obtained. As such aqueous media, for example a fibrinogen dissolving liquid and thrombin dissolving liquid (manufactured by The Chemo-Sero-Therapeutic Research Institute) and the like are shown as examples. In addition, the “aqueous liquid” in the present aspect means a state in which various components in the present aspect are dissolved and/or dispersed in an aqueous medium. It should be noted that “water” is water generally called pure water or ultrapure water in the medicinal and biotechnology field, which normally indicates water purified using at least one selected from ion exchange method, distillation method, reverse osmosis membrane method, and (reverse osmosis+continuous ion exchange method). The water is preferably ultrapure water, more preferably ultrapure water purified by ultrapure water system Milli-Q (product name) manufactured by Millipore (so-called Milli-Q water), but is not particularly restricted as long as a tissue adhesive, an object of the present aspect, can be obtained. The “pure water” generally indicates water which shows a specific resistivity of 1 MΩ·cm (25° C.) or more, preferably 3 MΩ·cm (25° C.) or more, and the “ultrapure water” indicates one which shows a specific resistivity of 15 MΩ·cm (25° C.) or more, preferably 18 MΩ·cm (25° C.) or more, and has an organic substance amount (TOC) of 50 ppb or less, preferably 20 ppb or less.

The concentration of fibrinogen is related to the concentration of adhesive for a living body, and it is thought that an adhesive having a larger concentration shows a higher adhesive power. In addition, thrombin has a catalytic action on the polymerization of fibrinogen, and thus fibrin glue formation is fast when the concentration of thrombin is large, and fibrin glue formation is slow when the concentration of thrombin is small. Therefore, the concentrations of fibrinogen and thrombin are appropriately selected depending on properties required for the tissue adhesive according to the present aspect (e.g. adhesive strength, time required for solidification, etc.)

The respective concentration of fibrinogen and thrombin in a first composition and a second composition is not particularly restricted, and the concentration of fibrinogen in the first composition is preferably 40 to 80 mg/mL. In addition, the concentration of thrombin in the second composition is preferably 250 to 300 unit/mL. The amount applied when applying these compositions to body tissues is also not particularly restricted, and conventionally known knowledge can be appropriately referred to. When using the above amount of first composition and second composition, an amount of about 0.5 to 2 mL each per 10 cm² body tissue (preferably about 1 mL each) can be applied.

In order that fibrin glue obtained by mixing fibrinogen and thrombin is allowed to form gel and function as glue, the pH of the fibrin glue is preferably a value from 5.5 or higher and 7.0 or lower. A means for adjusting the pH of fibrin glue to this range is not particularly restricted, and conventionally known knowledge can be appropriately referred to.

It should be noted that the above-described fibrin glue and a precursor thereof may contain pharmaceutically acceptable additives. Such additives can be directly contained in fibrin glue. Furthermore, in a case where the additives are contained in a precursor, they can be contained in the above-described first composition and/or second composition, and can be contained in other compositions, which are mixed with the first composition and second composition and used. Examples of such additives include for example blood coagulation factor XIII, trypsin inhibitors (aprotinin, etc.), albumin, collagen, polyglycolic acid (PGA), isoleucine, glycine, arginine, glutamic acid, surface active agents, pH adjusters, sodium chloride, calcium chloride, sugar alcohols (glycerol, mannitol, etc.), sodium citrate and the like. Among these, when blood coagulation factor XIII is contained, there is an advantage in being able to form a strong fibrin net. In addition, when a trypsin inhibitor such as aprotinin is contained, the digestion (decomposition) of the generated fibrin by a digestive enzyme such as trypsin can be suppressed.

As described above, a case where a tissue adhesive containing a protein is the so-called “fibrin glue” is provided as an example, and the details thereof are described. It should be noted, however, that in the method for attaching body tissues according to the present aspect, the tissue adhesive is not limited only to fibrin glue, and a tissue adhesive in which bovine serum albumin (BSA) is cross-linked using glutaraldehyde, and a tissue adhesive which is newly developed can be used. It should be noted that as a commercially available product of the tissue adhesive in which bovine serum albumin (BSA) is cross-linked using glutaraldehyde, for example, BioGlue Surgical Adhesive is known.

[Body Tissue]

In the method for attaching body tissues according to the present aspect, a specific site of the body tissue to which a tissue adhesive is applied is also not particularly restricted, and sites for which a tissue adhesive has been conventionally used can be suitably adopted. As an example of such sites applied, for example, a suture site after a surgical operation, a bleeding site, a fixed site of fracture pieces, an anastomotic site in a peripheral nerve and a microvessel, an affected part in tendon adhesion and tendon suture, an adhesion site in organ wound area, and the like are provided. Among these, as a site in which the problem of the present invention is more easily caused, a site which can be brought into contact with a protease such as a digestive enzyme is preferred. In particular, a suture site after a surgical operation for pancreas (secreting trypsin and chymotrypsin, which are proteases) is particularly preferred, and a suture site after a surgical operation for pancreatic cancer is most preferred. In other words, a preferred embodiment of the method for attaching body tissues according to the present aspect is a method for preventing and/or treating a pancreatic fistula.

[Heat Treatment]

The method for attaching body tissues according to the present aspect is characterized by the heat treatment of a protein contained in a tissue adhesive.

A specific embodiment for heat treatment is not particularly restricted, and a treatment by which the temperature of a protein contained in a tissue adhesive can be not less than a denaturation temperature of the protein can be suitably adopted. In a preferred embodiment, the temperature of the heat-treated protein contained in a tissue adhesive is 60 to 100° C., more preferably 70 to 100° C., and further preferably 80 to 90° C. According to the method for attaching body tissues according to the present aspect, a decrease in adhesive strength by a tissue adhesive can be suppressed by such heat treatment. A mechanism in which a decrease in adhesive strength is suppressed by the heat treatment is not completely obvious, and the present inventors presume that a protein contained in a tissue adhesive is denatured by the heat treatment and consequently the resistance of the protein (which is not easily digested) to protein digesting enzymes (trypsin, chymotrypsin, etc.) is improved. It should be noted that this mechanism is based on presumption and whether the mechanism is correct or not does not affect the scope of the present invention. From the results of Examples described below, it is obvious that the resistance of a tissue adhesive such as fibrin glue to digestive enzymes is improved by the heat treatment. Before confirming the results of Examples, the heat treatment of a tissue adhesive applied to a body tissue had been expected to decrease the effect of preventing enzyme leakage and to significantly decrease the adhesive strength (pressure resistance) because for example a protein forming the adhesive is not only denatured but also deteriorated and carbonated. However, such decrease in adhesive strength by heat treatment is not actually confirmed, or rather a surprising phenomenon in which the action of suppressing a decrease in adhesive strength is shown by an improvement in resistance to digestive enzymes was observed, thereby completing the present invention.

A means for heat treatment (heating means) is also not particularly restricted, and any known means can be suitably adopted as long as a protein contained in a tissue adhesive can be heated for example to the above-described suitable temperature. Examples of such heating means include an atmospheric pressure plasma treatment apparatus and moreover an electric knife without spark discharge. Among these, it is preferred that an atmospheric pressure plasma treatment apparatus be adopted as a heating means from the viewpoint that a better resistance to digestive enzymes can be provided to fibrin glue.

The “atmospheric pressure plasma” as used herein includes a pressure range around atmospheric pressure because “atmospheric pressure” includes the meaning of a higher pressure than vacuum plasma and low-pressure plasma and is figuratively used, and practically means plasma from about 0.1 to 10 atmospheres. The conditions when treating a body tissue using atmospheric pressure plasma are not particularly restricted and are only needed to be the condition that a protein contained in a tissue adhesive be able to be heated for example to the above-described suitable temperature.

It should be noted that as techniques relating to an apparatus for atmospheric pressure (low-temperature) plasma treatment (irradiation), for example JP 2015-76143 A, JP 2014-519875 W, WO 2012/005132 A can be referred to. In particular, when using an apparatus by which plasma can be generated on the point of a thin tubular electrode as described in JP 2015-76143 A, there are advantages that only an extremely localized site for example with ϕ1 mm can be heated and heat treatment by plasma irradiation can be certainly carried out even on an affected part in which the structure of a body tissue is intricate.

In addition, the “electric knife without spark discharge” as used herein is a treatment means which is so-called “SOFT coagulation” and as an example “Soft Coag mode” in an electric knife manufactured by ERBE (VIO system series) is provided. This mode is a special mode, and is characterized by having control different from that of conventional electric knives. That is, in the “Soft Coag mode” in the VIO series, the peak voltage is controlled to be 200 Vp or less with reference to the set output (W), and thus a coagulation effect can be caused to a body tissue without the generation of spark (spark discharge). At this time, electric energy is exchanged into heat in a metal portion, and as a result, the surface temperature of an apparatus is controlled to about 60 to 100° C. In the method for attaching body tissues according to the present aspect, by making use of energy for causing such coagulation effect, a protein can be heated so that the temperature of the protein contained in a tissue adhesive will be a value for example in the above predetermined range. Therefore, various conditions when heat treatment is carried out by using an “electric knife without spark discharge” can be appropriately set so that the temperature of a protein contained in a tissue adhesive will be a desired value (see also JP 2013-544122 W). It is preferred that, even when any means is adopted as a heating means, the surface temperature of a heat source in an apparatus be confirmed. A conventionally known temperature sensor can be suitably used as a means for such monitoring.

<<Kit for Preparing Tissue Adhesive>>

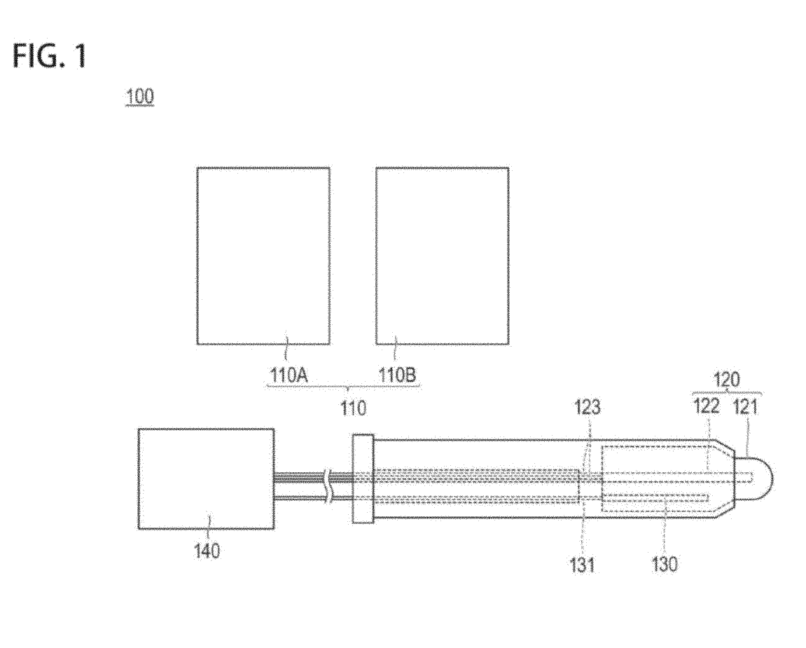

According to another aspect of the present invention, there is also provided a kit including the above-described “first composition 110A containing fibrinogen” and “second composition 110B containing thrombin” as essential components as shown in FIG. 1, as a kit for preparing a tissue adhesive, which is used for the method for attaching body tissues according to the above-described aspect of the present invention. The kit for preparing a tissue adhesive may further comprise, in addition to the first composition 110A and the second composition 110B liquids dissolving these compositions, other additives, an instruction manual, a packaging container, etc. The details of the first composition 110A and the second composition 110B, and the details of dissolving liquids and additives are as described above, and thus the detail descriptions are omitted here.

<<Device for Tissue Adhesion>>

According to yet another aspect of the present invention, there is also provided a device for tissue adhesion.

Such a device includes a kit for preparing a tissue adhesive, which contains a precursor of the tissue adhesive containing a protein which is applied to body tissues and used to attach the body tissues. As the composition of the kit for preparing a tissue adhesive, the composition of the kit for preparing a tissue adhesive according to the above-described aspect can be adopted in the same manner.

In addition, such a device further includes a heating means used for the heat treatment of the protein simultaneously with or after applying the tissue adhesive to the body tissues. As the heating means, any known means can be suitably adopted as long as a protein contained in the tissue adhesive can be heated for example to the above-described suitable temperature. Examples of such heating means include an atmospheric pressure plasma treatment apparatus and an electric knife without spark discharge, which are described in the section of the method for attaching body tissues according to the above-described aspect.

FIG. 1 is a figure for illustrating one embodiment of the above-mentioned device for tissue adhesion. As shown in the FIG. 1, the device 100 includes a kit for preparing tissue adhesive 110, heating means 120, a temperature detection part 130, and a temperature controller 140.

The kit for preparing tissue adhesion 110 contains a first composition 110A containing fibrinogen and a second composition 110B containing thrombin. The details of the first composition 110A and the second composition 110B, and the details of dissolving liquids and additives are as described above, and thus the detail descriptions are omitted here.

The heating means 120 includes an apical part 121 to contact with the object to be heated and to subject the object to heat treatment, and a bar-shaped heater 122 to heat the apical part 121. It is preferred that a coating of Teflon (registered trademark) is formed on the surface of the apical part 121. The heater 122 is connected to a temperature controller 140 via a power line 123.

The temperature detection part 130 contains a temperature detection element such as a thermocouple and a Peltier element to detect the temperature of the heater 122. The temperature detection part 130 is connected to the temperature controller 140 via a power line 131.

The temperature controller 140 controls the temperature of the heater 122 based on the data detected by the temperature detection part 130.

### EXAMPLES

The effect of the present invention will be described below by way of Examples and Comparative Examples. It should be noted, however, that the scope of the present invention is not restricted only to the Examples below.

### Example 1: Heat Treatment of Fibrin Glue by So-Called SOFT Coagulation

First, “BOLHEAL (registered trademark) for tissue adhesion” was prepared as a preparation kit for preparing fibrin glue, a tissue adhesive. Next, a solution A and a solution B were each prepared in accordance with the accompanying instruction manual.

Next, a polypropylene petri dish was prepared, and 200 μL of the A solution of BOLHEAL prepared above was added dropwise to the petri dish. On the added A solution, 200 μL of the B solution was added dropwise to form a fibrin clot.

In the present Example, the heat treatment of the fibrin clot formed above was carried out by using a heating means modeled on the so-called “SOFT coagulation”. Specifically, as such heating means, a bar-shaped (ϕ5 mm) heater in which temperature can be controlled by a temperature controller (Teflon (registered trademark) Coat made of C3604 (type II free-cutting brass)) was used. It should be noted that, when the preset temperature of the heater in such heating means was changed in a range from 60 to 140° C., the surface temperature of the heater was linearly changed from 40 to 70° C. Because the surface temperature of a heater can be equated with the temperature of the heat-treated body tissue, the preset temperature of a heater during the heat treatment was set to 110 to 130° C. so that the surface temperature of the heater would be 60 to 70° C. in the present Example . It should be noted that this heat treatment was carried out on both sides, the front and the back, in half of the region of the fibrin clot.

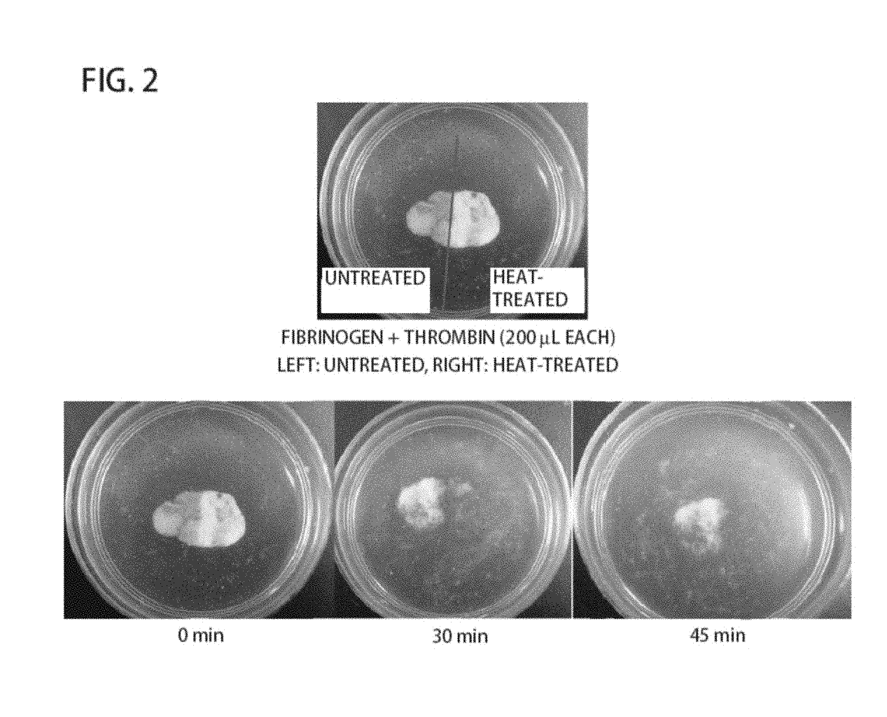

On the other hand, 10 mL of a solution obtained by adding and dissolving 1.5 g of sodium hydrogen carbonate and 1.0 g of pancreatin (derived from swine) in 100 mL of RO water was taken, and 0.3 g of pancreatin was further added thereto and dissolved, and thus pseudo-pancreatic fluid was prepared. In such pseudo-pancreatic fluid, the concentration of pancreatin was 4 w/v %, and the concentration of sodium hydrogen carbonate was 1.5 w/v %. Immediately after the above heat treatment, 5 to 6 mL of the pseudo-pancreatic fluid thus prepared was put in the petri dish containing the heat-treated fibrin clot. The digestive enzyme resistance to the pseudo-pancreatic fluid was evaluated in this manner between the heat-treated half region and the remaining non-heat-treated half region (see the upper side in FIG. 2). It should be noted that the environmental temperature in the experiment was maintained to the environmental temperature of an oven, 40° C.

As a result, as seen from the changes from the left photograph on the lower side (0 minutes after the heat treatment), the middle photograph (30 minutes after the heat treatment) to the right photograph (45 minutes after the treatment) in FIG. 2, the heat-treated region was not digested by the pseudo-pancreatic fluid even after 45 minutes from the heat treatment and the fibrin clot remained (the white region observed in the photographs; the fibrin clot turns 180° in the 30 and 45 min photographs). On the other hand, the non-heat-treated region was digested by the pseudo-pancreatic fluid, and thus it is found that a fibrin clot does not exist already. Therefore, it was demonstrated that the resistance of a fibrin clot to digestive enzymes could be improved by heat treatment.

### Example 2: Heat Treatment of Fibrin Glue by Atmospheric Pressure Plasma Irradiation

Unlike the Example 1, in the present Example, the heat treatment of a fibrin clot was carried out by using a plasma generator described in JP 2015-76143 A (which is an atmospheric pressure plasma irradiating apparatus) with the following irradiation conditions, in place of SOFT coagulation.

(Atmospheric Pressure Plasma Irradiation Conditions)
Input power supply: AC 100 V±5 V 50/60 Hz
High voltage supply voltage: 8 to 10 kVo-p
High voltage supply current: 25 to 31 mA (maximum)
Used gas: Helium (He) gas
Gas flow rate: 2 to 3 L/min.

In this method, a metal element of a thermistor was firstly irradiated with plasma and the temperature of plasma was measured. From the measurement results, the temperature was changed from 60 to 100° C. when the same point was irradiated with plasma for 10 to 30 seconds. Next, a fibrin clot was uniformly irradiated with plasma on the same conditions while moving irradiation so that irradiation would not focus on the same point in view of an increase in the temperature of the fibrin clot (the diameter of plasma flare: 1 mm). At this time, because the fibrin clot was denatured to whiten, it was judged that the fibrin clot was uniformly irradiated with plasma.

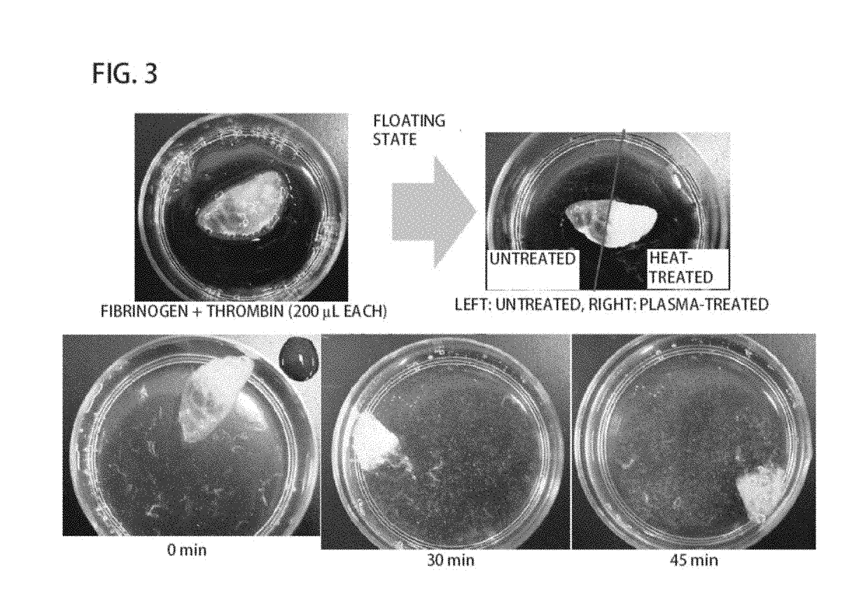

The heat treatment was carried out on both sides, the front and the back, in half of the region of a fibrin clot also in the present Example, and the plasma irradiation time at this time was 120 seconds on each of the front and the back (see the upper side in FIG. 3).

As a result, as seen from the changes from the left photograph on the lower side (0 minutes after the heat treatment), the middle photograph (30 minutes after the heat treatment), to the right photograph (45 minutes after heat treatment) in FIG. 3, the heat-treated region was not digested by the pseudo-pancreatic fluid even after 45 minutes from the heat treatment and the fibrin clot remained (the white region observed in the photographs). On the other hand, the non-heat-treated region was digested by the pseudo-pancreatic fluid, and thus it is found that a fibrin clot does not exist already. Therefore, it was demonstrated also in the present Example that the resistance of a fibrin clot to digestive enzymes could be improved by heat treatment.

### Example 3: Influence of Differences in Heating Means on Digestive Enzyme Resistance

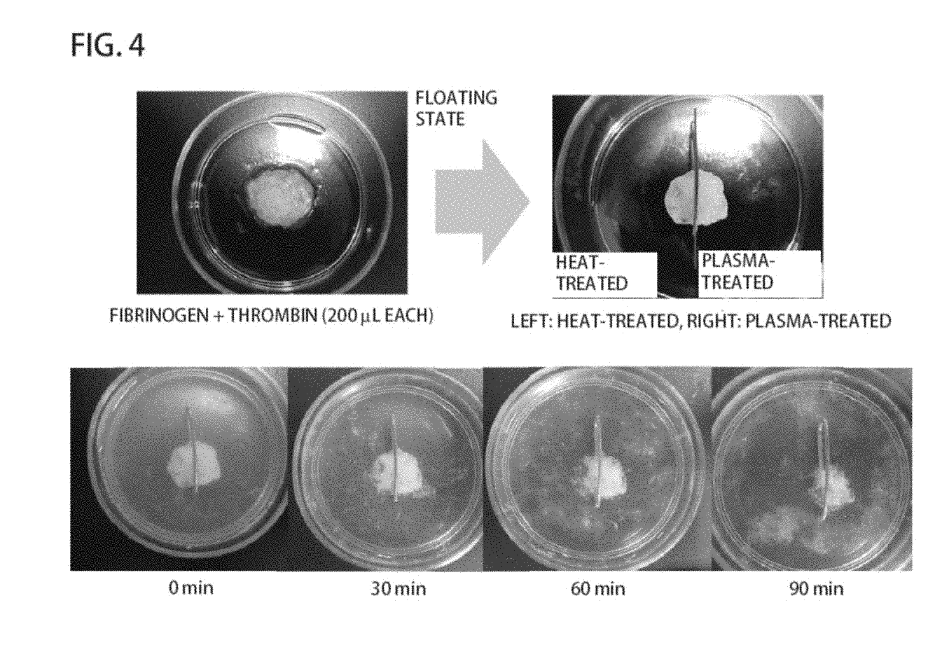

In the present Example, the influence of differences in heating means on the digestive enzyme resistance of fibrin clot was evaluated by comparing heating means each adopted in the Example 1 and the Example 2 described above. Specifically, heat treatment by SOFT coagulation (Example 1) was carried out on both sides, the front and the back, in half of the region of a fibrin clot soaked in the pseudo-pancreatic fluid in a petri dish. On the other hand, heat treatment by atmospheric pressure plasma irradiation (Example 2) was carried out on both sides, the front and the back, in the remaining half region (see the upper side in FIG. 4).

As a result, when comparing the changes in 4 photographs on the under side in FIG. 4 (0, 30, 60 and 90 minutes after the heat treatment from the left), differences were seldom observed until 30 minutes after the heat treatment, while the region of the SOFT coagulation was slightly digested by the pseudo-pancreatic fluid at 60 minutes after the heat treatment, and digestion in the region of heat treatment by SOFT coagulation was relatively significantly observed compared to that in the region of atmospheric pressure plasma irradiation at 90 minutes after the heat treatment. It is obvious, however, that a case of heat treatment is superior to a case where heat treatment is not carried out by the Example 1 and the Example 2 described above, and the concentration of the digestive enzyme in the pseudo-pancreatic fluid is set to be considerably higher than the concentration in an environment in which an actual body tissue is exposed. Therefore, an advantage of the heat treatment by SOFT coagulation is not denied also by the results of the present Example.

What is claimed is:

1. A method for attaching body tissues by applying a tissue adhesive comprising a protein to the body tissues,
the method comprising a heat treatment of the protein simultaneously with or after applying the tissue adhesive to the body tissues.

2. The method according to claim 1, wherein a temperature of the heat-treated protein is 60 to 100° C.

3. The method according to claim 1, wherein the heat treatment is carried out by using an atmospheric pressure plasma treatment apparatus or an electric knife without spark discharge.

4. The method according to claim 1, wherein the protein is fibrin.

5. The method according to claim 4, further comprising generating the fibrin by contact between
a first composition containing fibrinogen, and
a second composition containing thrombin.

6. The method according to claim 5, wherein
a concentration of fibrinogen in the first composition is 40 to 80 mg/mL, and
a concentration of thrombin in the second composition is 250 to 300 unit/mL.

7. The method according to claim 1, wherein the body tissue is a suture site after a surgical operation for pancreatic cancer.

8. A kit for preparing a tissue adhesive used for the method according to claim 1, the kit comprising:
a first composition containing fibrinogen; and
a second composition containing thrombin.

9. A device for tissue adhesion, the device comprising:
a kit for preparing a tissue adhesive, which contains a precursor of the tissue adhesive containing a protein which is applied to body tissues and used to attach the body tissues; and
a heating means used for a heat treatment of the protein simultaneously with or after applying the tissue adhesive to the body tissues.

10. The device for tissue adhesion according to claim 9, the device comprising a kit for preparing a tissue adhesive,
wherein the kit for preparing a tissue adhesive comprises a first composition containing fibrinogen, and
a second composition containing thrombin.
